(19) United States
(12) Plant Patent Application Publication
(10) Pub. No.: US 2003/0028942 P1
(43) Pub. Date: Feb. 6, 2003

(54) Gladiolus plant named 'White Knight'

(76) Inventor: John O. Zipperer III, Ft. Myers, FL (US)

Correspondence Address: LAURA G. BARROW, ESQ., P.O. BOX 215, ESTERO, FL, 33928-0215, US

(21) Appl. No.: 09/918,660

(22) Filed: Jul. 31, 2001

(51) Int. Cl. A01H 5/00

(52) U.S. Cl. PLT/301

(57) ABSTRACT

A new and distinct gladiolus variety, designated ‘White Knight’, is shown and described. Compared to the ‘T-200 variety, the ‘White Knight’ variety has a stem length that is about 10 cm longer, contains 1 to 2 more florets per flower head, and can hold open 1 to 2 more florets.

The present invention comprises a new and distinct variety of a Gladiolus plant referred to by the variety name ‘White Knight.’

### BRIEF DESCRIPTION OF THE PHOTOGRAPH

FIG. 1 is a photograph of a ‘White Knight’ variety plant in bloom.

### ASEXUAL REPRODUCTION

The new variety was originated by the Applicant in a controlled proprietary breeding program in Ft. Myers, Fla. wherein selected gladiolus varieties were crossed (the Applicant's selected provisional breeder's designation is 5-22). The female parent was a rich purple gladiolus variety named ‘Dr. Magee,’ characterized in part by having a small pink bloom, a short stem having a short flower head, and high resistance to Fusarium fungi species. The male parent was a white gladiolus variety named ‘T-200,’ an unpatented, released variety by Turk characterized in part by having a harvesting time similar to the ‘White Friendship’ gladiolus variety, 16 to 18 florets, and lightweight stems. The seeds were planted in Ft. Myers, Fla., and the selection of the ‘White Knight’ variety was made in Spring 1988. Asexual reproduction of the ‘White Knight’ variety was achieved by collecting cormels from the first corm. All subsequent asexual reproductions of the ‘White Knight’ variety remain true to the original variety type.

Botanical Description:

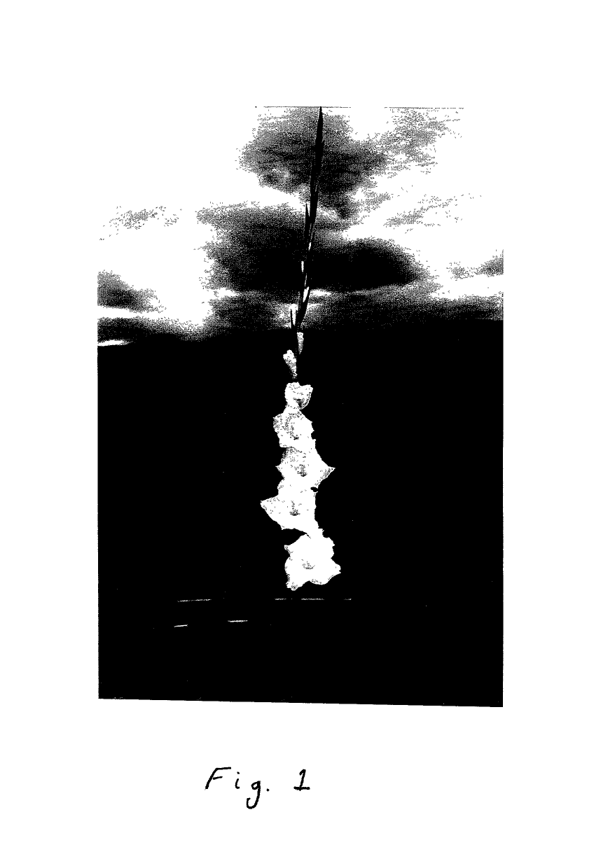

The accompanying color photograph (FIG. 1) shows the inflorescence and various stages of blooming of the ‘White Knight’ variety plant.

The bud size of the ‘White Knight’ variety is about 8 cm in length. The flowers of the variety each consist of a total of six petals, namely three large petals located on the top half of the flower, a medium-size petal positioned on the lower half of the flower (at 6:00), and two small petals also located on the lower half of the flower (at 4:00 and 8:00). Some flowers of the variety have been observed having five large petals and one small petal. The variety has a light green color deep in the throat, followed by a pure white color throughout the rest of the petals.

The spike of the plant is about 124 cm and contains 18-19 florets when grown from Jumbo size corms (1.75 inch and larger) in Ecuador. The head length is about 58 cm, and the bloom diameter is 12 cm. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes). The pistils of the flower are white, and the stamens are tannish yellow.

The corms of the ‘White Knight’ variety are typical for the gladiolus.

The leaf color of the ‘White Knight’ variety is green. The leaf size is about 70 cm (length) by 3.2 cm (width).

General Observations:

Both the ‘White Knight’ variety and ‘T-200 variety take about 76 days to harvest in Ecuador. Both the ‘White Knight’ and ‘T-200’ varieties prefer longer day lengths, do not grow well in the mid-winter, have good tolerance to heat, and have good resistance to bacterial neck rot.

The variety usually produces one to two stems per Jumbo size corm. The variety can maintain six to seven florets in open bloom simultaneously in a vase of water and can be harvested with one flower showing color. The variety is an excellent opener after transport, in that the stems may be cut tight, shipped dry for a week, and still bloom well afterwards.

The ‘White Knight’ variety is much more resistant to attack by Fusarium fungi than the ‘T-200 variety. Unlike the ‘T-200’ variety, the ‘White Knight’ variety does not have the tendency of producing blind stems when a bulb has been used two to three times for subsequent asexual reproductions.

Compared to the ‘T-200 variety, the ‘White Knight’ variety contains 1 to 2 more florets per flower head, can hold open 1 to 2 more florets, has a stem length that is about 10 cm longer, has a greater stem diameter, and has a flower diameter that is 1 to 1.5 cm larger. Also, unlike the ‘T-200’ variety, the bottom floret of the ‘White Knight’ variety often protrudes out to one side more.

I claim:

1. A new and distinct gladiolus variety ‘White Knight’, as shown and described herein.
